(19) United States
(12) Patent Application Publication
(10) Pub. No.: US 2023/0131115 A1
(43) Pub. Date: Apr. 27, 2023

(54) System and Method for Displaying Position of Echogenic Needles

(71) Applicant: GE Precision Healthcare LLC, Wauwatosa, WI (US)

(72) Inventors: Menachem Halmann, Monona, WI (US); Alex Sokulin, Kiryat Tivon (IL); Dani Pinkovich, Atlit (IL); Cynthia A. Owen, Powhatan, AR (US)

(21) Appl. No.: 17/507,451

(22) Filed: Oct. 21, 2021

(51) Int. Cl. A61B 8/08; A61B 8/00; G06K 9/62; G06T 7/73; G16H 30/20

(52) U.S. Cl. CPC A61B 8/0841; A61B 8/463; A61B 8/481; G06K 9/6202; G06T 7/74; G16H 30/20; G06T 2207/10132

(57) ABSTRACT

A system and method is provided for providing an indication of viewable and non-viewable parts of an interventional device in an ultrasound image. The system includes a processing unit including a detection and recognition system configured to detect a pattern of echogenic features within ultrasound images, and a memory unit operably connected to the processing unit storing information regarding echogenic patterns on individual interventional devices. The detection and recognition system determines viewable and non-viewable parts of detected echogenic patterns in the ultrasound image by comparing the dimensions of the stored echogenic patterns with the representation of the detected echogenic patterns in the ultrasound images and positions an indicator within the ultrasound image on the display in alignment with the locations of the viewable and non-viewable parts of the detected echogenic patterns on the interventional device.

### BACKGROUND OF THE INVENTION

The present disclosure is generally directed to interventional medical articles (for example, needles, catheters, cannulas, sheaths, etc.) including features that provide enhanced ultrasound visibility during introduction and/or delivery into a body space, such as, for example, an artery, vein, vessel, body cavity, or drainage site, and more specifically directed to systems and methods for determining the location of the medical article within the body of a patient.

Ultrasonic imaging is used to examine the interior of living tissue and the image is used to aid in the performance of medical procedures on this tissue. One such procedure is the insertion of an interventional device, such as a needle to a desired location in the tissue, for instance the insertion of a needle into a lesion or other anomaly in the tissue to take a biopsy, or to inject the tissue with a diagnostic or medical treatment material, such as a local anesthesia or nerve block. As the needle is inserted into the body of the patient, ultrasonic imaging is performed in conjunction with the insertion of the needle to illustrate on an associated display the position of the needle within the body of the patient relative to the tissue that is the target for the insertion of the needle.

In order to safely and effectively perform the procedure employing the needle, it is necessary to be able to determine the exact location of the tip of the needle in order to direct the tip into the desired area of the tissue that is the subject of the procedure. However, in some cases the entire body of the needle and particularly the tip of the needle is not readily apparent in the ultrasound image. For example, during insertion the tip of the needle may be inadvertently directed or deflected out of the imaging plane for the ultrasonic images being obtained. As a result, only the portion of the needle body behind the tip that remains in the imaging plane is visible in the displayed ultrasound image, while the actual position of the tip of the needle is disposed ahead of the portion of the needle that is visible in the displayed ultrasound image. Thus, with this displayed ultrasound image, the user may think the portion of the needle illustrated in the ultrasound image defines the proper location of the tip of the needle, such that the user can potentially cause unintentional damage to other organs or unintended injections into vessels with the further insertion of the needle into the body of the patient.

In the prior art, to enhance the ability of the ultrasound imaging system to provide an accurate display of the position of the needle including the needle tip within the body of the patient, needles have been developed that include an echogenic portion on the needle, such as those examples disclosed in US Patent Application Publication Nos. US2017/0043100, entitled Echogenic Pattern And Medical Articles Including Same, and US2012/0059247, entitled Echogenic Needle For Biopsy Device, the entirety of which are hereby expressly incorporated herein by reference for all purposes. In certain needles, the echogenic portion of the needle can be formed adjacent the tip of the needle in order to provide enhancement to the ultrasound imaging of the tip as it is inserted into the body of the patient.

However, even with the enhanced echogenic features disposed on the needle, it is still possible for the tip of the needle including the echogenic features to be directed or deflected out of the imaging plane. In that situation, the user may still view the ultrasound image showing less than the entirety of the needle and may inadvertently further insert the needle into the patient creating a highly undesirable situation.

Therefore, it is desirable to develop a system and method for the ultrasonic imaging of a needle inserted into the body of a patient that can provide the user with an accurate indication of the location of the tip of the needle when the needle tip is deflected or directed out of the imaging plane for the ultrasonic imaging system.

### BRIEF DESCRIPTION OF THE DISCLOSURE

In one exemplary embodiment of the invention, an ultrasound imaging system for obtaining ultrasound images of an interior of an object includes a processing unit configured to receive and process acquired ultrasound image data to create ultrasound images derived from ultrasound image data, the procession unit including a detection and recognition system configured to detect a pattern of echogenic features within the created ultrasound images, a memory unit operably connected to the processing unit and storing information regarding individual interventional devices, including stored echogenic patterns and specific locations and dimensions of the stored echogenic patterns on the individual interventional devices, a display operably connected to the processing unit to present the ultrasound images to a user, an ultrasound imaging probe operably connected to the processing unit to acquire the ultrasound image data for use by the processing unit to form the ultrasound images, wherein the detection and recognition system is configured to detect a pattern of echogenic features within the ultrasound image data, to compare a detected echogenic pattern to the stored echogenic patterns within the memory unit and to position an indicator within an ultrasound image on the display illustrating a position of viewable and non-viewable parts of the detected echogenic pattern.

In another exemplary embodiment of the invention, an ultrasound imaging system includes a processing unit configured to receive and process acquired ultrasound image data to create ultrasound images derived from ultrasound image data, the procession unit including a detection and recognition system configured to detect a pattern of echogenic features within the created ultrasound images, a memory unit operably connected to the processing unit and storing information regarding individual interventional devices, including stored echogenic patterns and specific locations and dimensions of the stored echogenic patterns on the individual interventional devices, a display operably connected to the processing unit to present the ultrasound images to a user, an ultrasound imaging probe operably connected to the processing unit to acquire the ultrasound image data for use by the processing unit to form the ultrasound images, and an interventional device adapted to be inserted into an object adjacent the imaging probe, the interventional device including one or more echogenic portions thereon, the one or more echogenic portions including one or more echogenic features disposed thereon forming one or more echogenic patterns, wherein the detection and recognition system is configured to detect one or more echogenic patterns within the ultrasound image data, to compare a detected echogenic pattern to the stored echogenic patterns within the memory unit and to position an indicator within an ultrasound image on the display illustrating a position of viewable and non-viewable parts of the detected echogenic pattern.

In still another exemplary embodiment of the method of the invention, a method for providing an indication of viewable and non-viewable parts of an interventional device in an ultrasound image including the steps of providing an ultrasound imaging system having a processing unit configured to receive and process acquired ultrasound image data to create ultrasound images derived from ultrasound image data, the procession unit including a detection and recognition system configured to detect a pattern of echogenic features within the created ultrasound images, a memory unit operably connected to the processing unit and storing information regarding individual interventional devices, including stored echogenic patterns and specific locations and dimensions of the stored echogenic patterns on the individual interventional devices, a display operably connected to the processing unit to present the ultrasound images to a user, an ultrasound imaging probe operably connected to the processing unit to acquire the ultrasound image data for use by the processing unit to form the ultrasound images, and an interventional device adapted to be inserted into an object adjacent the imaging probe, the interventional device including one or more echogenic portions thereon, the one or more echogenic portions including one or more echogenic features disposed thereon forming one or more echogenic patterns, wherein the detection and recognition system is configured to detect one or more echogenic patterns within the ultrasound image data, to compare a detected echogenic pattern to the stored echogenic patterns within the memory unit and to position an indicator within an ultrasound image on the display illustrating a position of viewable and non-viewable parts of the detected echogenic pattern, inserting the interventional device into the object, obtaining ultrasound image data using the probe, matching one or more detected echogenic patterns to corresponding stored echogenic patterns in the memory unit, determining the dimensions of the stored echogenic patterns from the memory unit, determining the viewable and non-viewable parts of the one or more detected echogenic patterns by comparing the dimensions of the stored echogenic patterns with a representation of the one or more detected echogenic patterns in the ultrasound image data, and positioning the indicator within the ultrasound image on the display in alignment with the viewable and non-viewable parts of the one or more detected echogenic patterns on the interventional device.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 is a schematic view of an ultrasound imaging system according to an embodiment of the disclosure.

FIG. 2 is a schematic view of an ultrasound imaging system including an echogenic needle display system constructed according to an exemplary embodiment of the disclosure.

FIG. 3 is a front plan view of a first embodiment of an echogenic needle utilized with the needle display system of FIG. 2.

FIG. 4 is a cross-sectional view along line 4-4 of FIG. 3.

FIG. 5 is a front plan view of a second embodiment of an echogenic needle utilized with the needle display system of FIG. 2.

FIG. 6 is a schematic representation of an ultrasound image illustrating the detected position of an echogenic needle within the body of a patient.

FIG. 7 is a schematic representation of an ultrasound image illustrating the detected position of an imaged portion of an echogenic needle and estimated position of a non-imaged portion of the echogenic needle within the body of a patient.

FIG. 8 is a flowchart illustrating the method according to an exemplary embodiment of the present disclosure.

### DETAILED DESCRIPTION

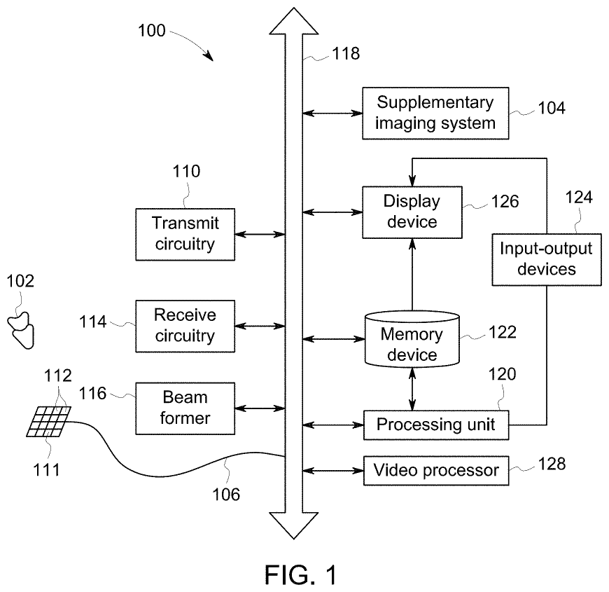

Referring to FIG. 1, illustrates an exemplary ultrasound imaging system 100 for use during ultrasound imaging procedures that includes an ultrasound probe 106, such as a linear array probe, for optimal visualization of a target structure 102 within a patient 20. The ultrasound imaging system 100 includes transmit circuitry 110 configured to generate a pulsed waveform to operate or drive a transducer array 111 including one or more transducer elements 112 disposed within the probe 106, and receive circuitry 114 operatively coupled to a beamformer 116 and configured to process the received echoes and output corresponding radio frequency (RF) signals.

Further, the system 100 includes a processing unit 120 communicatively coupled to the transmit circuitry 110, the beamformer 116, the probe 106, and/or the receive circuitry 114, over a wired or wireless communications network 118. The processing unit 120 may be configured to receive and process the acquired image data, for example, the RF signals according to a plurality of selectable ultrasound imaging modes in near real-time and/or offline mode.

Moreover, in one embodiment, the processing unit 120 may be configured to store the acquired volumetric images, the imaging parameters, and/or viewing parameters in a memory device 122. The memory device 122, for example, may include storage devices such as a random access memory, a read only memory, a disc drive, solid-state memory device, and/or a flash memory. Additionally, the processing unit 120 may display the volumetric images and or information derived from the image to a user, such as a cardiologist, for further assessment on a operably connected display 126 for manipulation using one or more connected user input-output devices 124 for communicating information and/or receiving commands and inputs from the user, or for processing by a video processor 128 that may be connected and configured to perform one or more functions of the processing unit 120. For example, the video processor 128 may be configured to digitize the received echoes and output a resulting digital video stream on the display device 126.

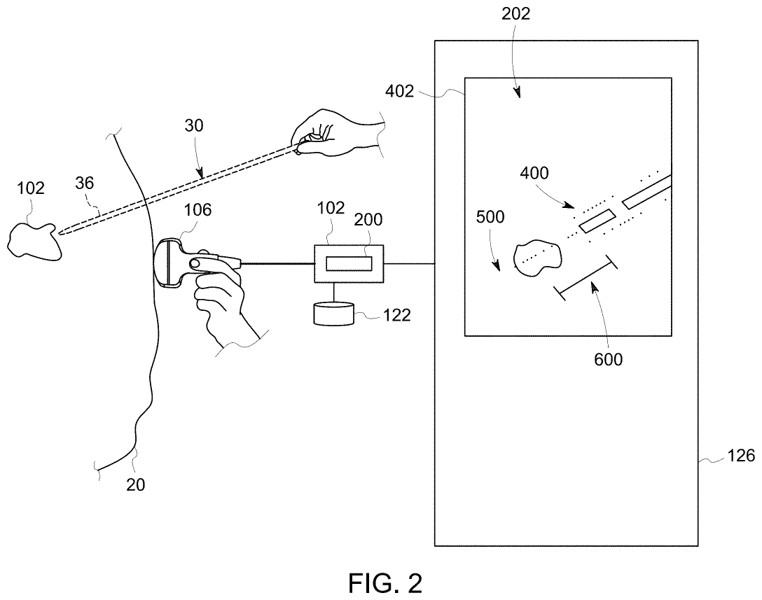

Referring now to FIG. 2, in use the probe 106 is placed adjacent to the patient 20 to provide ultrasound images of the target structure or tissue 102 within the patient 20. An interventional device 30 is mounted to or disposed adjacent the probe 106 and is adapted to be inserted into the patient 20 to the target tissue 102 either manually or through the use of a suitable insertion mechanism 36 operably connected to the device 30, and optionally to the probe 106. The interventional device 30 is shown in the illustrated exemplary embodiment as a needle 32, but in other embodiments can be another interventional device, such as a catheter, dilator or sheath, among others. The needle 32 includes one or more echogenic features 34 thereon to improve visibility of the needle 32 and portions thereof within ultrasound images. In certain exemplary embodiments, the needles 32 according to the present disclosure can be employed for the introduction or delivery of a medical material, such as local anesthesia or a nerve block, or another medical article, such as a catheter, cannula, or sheath, into a space, such as a blood vessel or drainage site. In other embodiments, the needles 32 according to the present disclosure can be used for biopsy or tissue sampling purposes. In any embodiment for the use of the device 30/needle 32, the echogenic features 34 can be formed on, in or added to the structure of the device 30/needle 32, e.g., coatings, glass beads, spherical particles, grooves, indentations or other features alone or in combination with one another that do not interfere with the function of the needle 32.

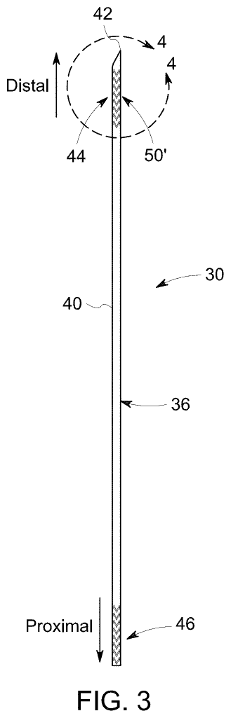

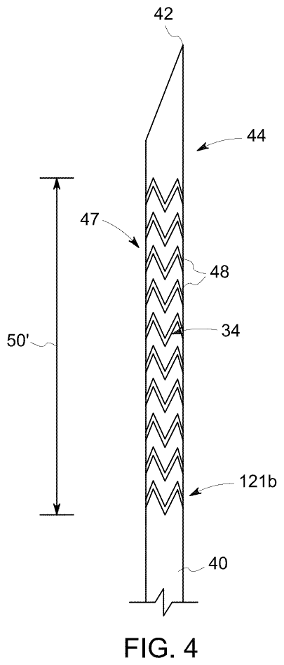

In the exemplary illustrated embodiment of FIGS. 3 and 4, the needle 32 includes a hollow, elongate body 40 having a tip 42 at a distal end 44, and a proximal end 46 opposite the tip 42. In the illustrated exemplary embodiment, the echogenic features 34 are positioned at and/or adjacent the tip 42, such that the tip 42 is provided with enhanced visibility in ultrasound images 202 obtained by the ultrasound imaging system 100. The echogenic features 34 are formed in the body 40 to have a pattern 47 for the features 34 that enables the features 34 to be readily viewed and distinguished from other structures located within the ultrasound images 202 obtained by the system 100. In the embodiment of FIG. 4, the echogenic features 34 take the form of grooves 48 etched into the material forming the body 40 of the needle 32 that are spaced from one another along an echogenic portion 50 of the body 40 of the needle 32.

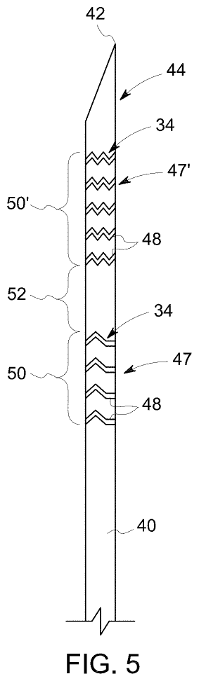

Alternatively, as shown in the illustrated exemplary embodiment of FIG. 5, the needle 32 can have a body 40 with a number of echogenic portions 50,50′ spaced from one another along the body 40. The echogenic portions 50 can have the same or different shapes and/or types of echogenic features 34 thereon, in order for the different portions 50,50′ to present specific viewable patterns 47,47′ in ultrasound images of the body 40. The echogenic portions 50 can be separated by bands 52 of the body 40 that do not include any echogenic features 34 thereon. As a result, the echogenic portions 50,50′ and patterns 47,47′ formed therein and the bands 52 enable the needle 32 to provide information through the ultrasound images regarding the position of the tip 42 of the needle 32 relative to one or more of the echogenic portions 50,50′ disposed on the body 40 of the needle 32.

Referring now to FIG. 2, the ultrasound imaging system 100 includes a detection and recognition system 200. The detection and recognition system 200 can be formed as a part of the processing unit 120 or can be a separate component of the ultrasound imaging system 100 that is operably connected to the processing unit 120. In either embodiment, the detection and recognition system 200 is configured to analyze the ultrasound image 202 produced by the processing unit 102 from the acquired image data in order to locate the presence of the needle 32 or other echogenic interventional device within the ultrasound image 202. The ultrasound images 202 can be individual 2D or 3D images or 2D or 3D frames within an 4D ultrasound video or cine loop. The detection and recognition system 200 is operably connected to the memory device 122 or to a separate electronic storage device or database (not shown) that contains information relating to the patterns 47,47′ of the echogenic features 34 for a number of different interventional devices 30/needles 32 from various manufacturers.

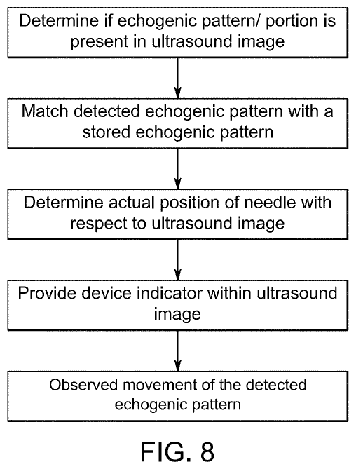

Looking now at FIGS. 2 and 6-8, when analyzing a particular ultrasound image 202 and/or the image data utilized to form the image 202 or a particular frame of a video or cine loop corresponding to the image 202, in block 300 the detection and recognition system 200 determines for each video or cine frame or ultrasound image 202 if an echogenic portion 50 and associated pattern 47 of a needle 32 is present within the ultrasound image 202. To detect the echogenic portion 50, the detection and recognition system 200 employs a suitable process to minimize noise within the ultrasound image data/ultrasound image 202 and enable any echogenic portion 50 and pattern 47 to be more readily located. In one exemplary embodiment for the detection and recognition system 200, the detection and recognition system 200 employs a suitable pattern-recognition algorithm, such as an algorithm utilizing matched filters in a known manner, and/or artificial intelligence (AI) located within the detection and recognition system 200 to determine the presence of the pattern 47 of any echogenic portion 50 within the image data/ultrasound image 202.

In an alternative exemplary embodiment, as a substitute for or a supplement to the automatic detection of the pattern 47 by the detection and recognition system 200 to identify the needle 32, the user interface/input device 124 can be operated to allow the user to select the type of needle 32 that is to be used in the procedure. The detection and recognition system 200 can then identify the pattern 47 for the needle 32 selected by the user and operate to locate that pattern 47 within the image data/ultrasound image 202. This information on the needle 32 to be used can be supplied to the detection and recognition system 200 by the user in various manners, such as through the input device 124, such as by manually entering identifying information on the needle 32, or by scanning a barcode or RFID located on packaging for the needle 32 including the identifying information.

If one or more echogenic portions 50 are determined to be present in the ultrasound image data/image 202, in block 302 the detection and recognition system 200 accesses the memory unit 122 containing the stored information on the different patterns of echogenic features associated with particular interventional devices 30/needles 32. The pattern 47 of the echogenic features 34 disposed on the echogenic portion(s) 50 detected by the detection and recognition system 200 is compared to the stored patterns in order to match the detected pattern 47 to the pattern utilized on a particular interventional device 30/needle 32.

Once the pattern 47 of the echogenic portion 50 detected in the ultrasound image data/image 202 is recognized and/or matched with a particular manufacturer, in block 304 the information stored in the memory unit 122 regarding the specific configuration of the particular interventional device 30/needle 32 including the recognized pattern 47 can be employed by the detection and recognition system 200 to determine the position of the needle 32 in relation to the ultrasound image 202. This is accomplished by the recognition and detection system 200 by comparing the location and/or dimensions (e.g., length) of the echogenic portion(s) 50,50′ and associated pattern(s) 47,47′ detected in the ultrasound image 202 and associated with a particular needle 32 with the dimensions of the needle 32 stored in the memory unit 122. For example, if the needle 32 detected in the ultrasound image 202 includes two echogenic portions 50,50′ spaced from one another by a band 52, with the echogenic portion(s) 50,50′ and the band 52 each having a specified length, the recognition and detection system 200 can determine the length of the body 40 of the needle 32 that is present within the image 202 based on the length of the echogenic portion(s) 50,50′ and band(s) 52 visible in the image 202.

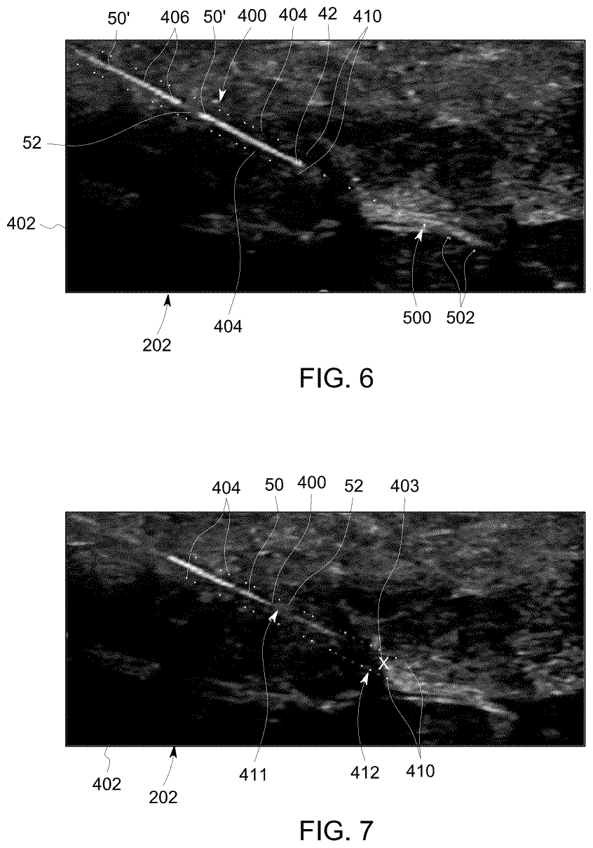

Using this information, in block 306 the recognition and detection system 200 can provide an enhancement to the representation of the needle 32 within the ultrasound image 202. Referring to FIGS. 6-8, a device indicator 400 provided by the determination and recognition system 200 can display information to the user within the frame 402 of the ultrasound image 202 represented on the display 126 concerning the location and orientation of the needle 32, and in particular the tip 42 of the needle 32, with regard to the image plane/frame 402 for the images 202 being obtained using the ultrasound imaging system 100. More specifically, knowing the relationship and/or distance of the echogenic portion(s) 50 and/or band(s) 52 visible in the image data/image 202 from the tip 42 of the needle 32, the recognition and detection system 200 can determine the location of the tip 42 relative to the image 202, even if the tip 42 is not viewable within the ultrasound image 202. With this location information, the detection and recognition system 200 can provide the device indicator 400 within the ultrasound image 202 regarding both the visible portions of the needle 32 and the portions of the needle 32 that are not visible in the image 202 as a result of being positioned out of the image plane/frame 402 for the ultrasound image 202.

For example, as illustrated in the exemplary embodiment of FIG. 6, the needle 32 being inserted into the patient 20 includes a pair of echogenic portions 50, 50′ spaced from one another by a single band 52, with the foremost echogenic portion 50′ terminating at the tip 42 for the needle 32. The information stored in the memory unit 122 regarding the length of the various parts of the needle 32, such as the first echogenic portion 50′ for the particular needle 32 is known and can be used by the detection and recognition system 200 to determine what length of the first echogenic portion 50′ is visible within the ultrasound image 202.

If the length of the first echogenic portion 50′ stored in the memory unit 122 corresponds to the length of the first echogenic portion 50′ represented in the ultrasound image 202, the detection and recognition system 200 can provide the device indicator 400 illustrating that the entirety of the first echogenic portion 50′ and the tip 42 are visible within the ultrasound image 202.

Conversely, if the system 200 determines that the length of the first echogenic portion 50′ stored in the memory unit 122 does not correspond to the length of the first echogenic portion 50′ represented in the ultrasound image 202, the detection and recognition system 200 can provide a device indicator 400 illustrating that a portion of the first echogenic portion 50′ and the tip 42 are outside of the image plane/frame 402 represented in the ultrasound image 202.

As shown in the illustrated exemplary embodiment of FIGS. 6 and 7, if the tip 42, and thus the entire first echogenic portion 50′ are viewable within the ultrasound image 202 as determined by the system 200 through the comparison of the viewable positions of the needle 32 with the known dimensions of the needle 32 and portions 50,50′ thereon, the device indicator 400 can take the form of a pair of boundary lines 404 located on each side of the needle 32 as shown in the ultrasound image 202. The boundary lines 404 are spaced on either side of the representation of the needle 32 in the ultrasound image 202 and extend along the entire length of the needle 32 that is shown in the ultrasound image 202, with the ends 410 of the boundary lines 402 positioned in alignment with the tip 42 of the needle 32. The boundary lines 404 can have any desired form and in the illustrated exemplary embodiment are formed by a number of equidistant spaced dots 406 aligned with representation of the needle 32 in the ultrasound image 202. In addition, as the needle 32 is inserted further into the patient 20, the detection and recognition system 200 can lengthen the boundary lines 402 to correspond to the length of the needle 32 represented within the ultrasound image 202 and maintain the alignment of the ends 410 with the tip 42 of the needle 32 as shown in the ultrasound image 202.

Alternatively, in the situation where the detection and recognition system 200 determines that less than the entire length of the first echogenic portion 50′ is represented or viewable within the ultrasound image 202, illustrating that the tip 42 has been directed and/or deflected out of the imaging plane for the image 202, the system 200 will position the boundary lines 404 along each side of the needle 32 represented in the ultrasound image 202. However, in this situation the boundary lines 404 presented by the system 202 will extend beyond the length of the needle 32 represented in the ultrasound image 202 to correspond in length and position to the actual position of the tip 42 of the needle 32 as determined by the detection and recognition system 200. As shown in the exemplary illustrated embodiment of FIG. 7, the boundary lines 404 extend past the representation of the needle 32 to the estimated point where the tip 42 of the needle 32 is actually positioned as determined by the system 200, thus visually illustrating the position of the entire needle 32 including the tip 42 relative to the actual position of the first portion of the needle 32 that remains viewable in the ultrasound image 202 and bounded by a first portion 411 of the boundary lines 404. In this manner, the detection and recognition system 200 not only provides the user with a position of the needle tip 42 based on the position of the ends 410 of the boundary lines 404 in the ultrasound image 202 based on their alignment with the tip 42 as determined by the system 202, but additionally indicates an estimation of the length of the needle 32 that extends out of the image plane for the ultrasound image 202 as represented by the second portion 412 of the boundary lines 402 extending between the ends 410 of the boundary lines 404 and the foremost viewable part of the needle 32 within the ultrasound image 202.

Further, in block 308 these first portions 411 and second portions 412 can be altered in orientation and/or length by the system 200 as the tip 42 of the needle 32 is moved, e.g., closer to or further away from the plane of the ultrasound image 202, as determined by the system 200 based on the portion(s) 50,50′ and/or band(s) 52 of the needle 32 that are viewable within the ultrasound image 202. As the ultrasound images 202 are presented to the user on the display 126, which can be done in a user-controlled frame rate or cine manner, or as a real-time, video display of the current position and movement of the needle 32 within the patient 20, the detection and recognition system 200 can alter the device indicator 400/boundary lines 404 to reflect the real time position of the needle 32 and tip 42 within and/or relative to the frame/image plane 402 represented by the images 202.

In addition to the length of the portions 411,412 of the boundary lines 404, the detection and recognition system 200 can enhance the indication of the location of the tip 42 out of the plane 402 of the ultrasound image 202 using the boundary lines 404. For example, the system 200 can change or add color to the portions 412 of the boundary lines 404 that is different from that for the first portions 411, such as by changing the color of those dots 406 forming the second portions 412 of the boundary lines 404 as shown in FIG. 7. Other alterations to the form of the boundary lines 404 and in particular the second portions 412 are contemplated to enhance the representation, such as by enlarging the size of the second portions 412 of the boundary lines 404.

As best shown in FIG. 6, the recognition and detection system 200 can also place a trajectory or path indicator 500 within the ultrasound image 202. The path indicator 500 is disposed in alignment with the long axis of body 40 of the needle 32 and represents the path the needle 32 will follow if inserted further into the patient 20 in a straight line. With the path indicator 500, the user can identify if the insertion path of the needle 32 is aligned with the tissue 102 intended to be intersected by the needle 32 in order to perform the desired medical procedure utilizing the needle 32. The illustrated exemplary embodiment shows the path indicator 500 in FIG. 6 represented as a line of dots 502 disposed in alignment with the body 40 of the needle 32 and in alignment with one another in order to enable the path indicator 500 to provide information concerning the projected straight-line path for further insertion of the needle 32 into the patient without obscuring any significant portions of the tissue 102 of the patient 20 represented within the ultrasound image 202. Further, while the line of dots 502 in FIG. 6 represents one exemplary embodiment for the path indicator 500, the form of the path indicator 500 can be selected as desired. Also, the path indicator 500 can be presented within the ultrasound image 202 as shown in FIG. 6 so long as the tip 42 of the needle 32 is determined to be within the ultrasound image 202. Thus, in the situation where the tip 42 has been directed outside of the image plane/frame 402 and thus outside of the ultrasound image 202, the detection and recognition system 202 can cease displaying the path indicator 500 as a device indicator 400 of the misalignment of the tip 42 that is separate from, or optionally utilized in place of the portions 411,412/boundary lines 404.

In addition to the device indicator 400/boundary lines 402 and path indicator 500, the detection and recognition system 200 can directly enhance the representation of the tip 42 within the ultrasound image 202 based on the fact that the position of the tip 42 is now known. More specifically, in one exemplary embodiment, the detection and recognition system 200 can brighten the representation and/or the expected area or location of the tip 42 within the ultrasound image 202 or provide another icon 403 aligned with the position of the tip 42 within the ultrasound image 202 as determined by the system 200. Alternatively, the detection and recognition system 200 can detect the motion of the tip 42 to brighten it, such as by changing some scan parameters in the area of the image data/ultrasound image 202 where motion of the tip 42 was detected to achieve higher resolution in that area.

With the system additionally being provided with the exact location of the target tissue 102 within the patient 20, the system 200 can also provide information on the display 126 regarding the distance between the tip 42 and the target tissue 102, such as a line extending between the tip 42 and tissue 102 and/or a real time measurement 600 (FIG. 2) of the current distance between the tip 42 and the target tissue 102.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. An ultrasound imaging system for obtaining ultrasound images of an interior of an object, the ultrasound imaging system comprising:
a processing unit configured to receive and process acquired ultrasound image data to create ultrasound images derived from ultrasound image data, the processing unit including a detection and recognition system configured to detect a pattern of echogenic features within the ultrasound image data;
a memory unit operably connected to the processing unit and storing information regarding individual interventional devices, including stored information regarding echogenic patterns and specific locations and dimensions of stored echogenic patterns on the individual interventional devices;
a display operably connected to the processing unit to present the ultrasound images to a user;
an ultrasound imaging probe operably connected to the processing unit to acquire the ultrasound image data for use by the processing unit to form the ultrasound images,
wherein the detection and recognition system is configured to detect the pattern of echogenic features within the ultrasound image data, to compare a detected echogenic pattern to the stored echogenic patterns within the memory unit and to position an indicator within an ultrasound image on the display illustrating a location of viewable and non-viewable parts of the interventional device in view of the positions of viewable and non-viewable parts of the detected echogenic pattern.

2. The ultrasound imaging system of claim 1, wherein the detection and recognition system comprises a pattern recognition algorithm.

3. The ultrasound imaging system of claim 1, wherein the detection and recognition system comprises a pattern recognition artificial intelligence.

4. The ultrasound imaging system of claim 1, wherein the detection and recognition system comprises a matched filter pattern recognition algorithm.

5. The ultrasound imaging system of claim 1, wherein the indicator comprises:
a first portion illustrating the position of viewable parts of the interventional device; and
a second portion illustrating the position of non-viewable parts of the interventional device.

6. The ultrasound imaging system of claim 5, wherein the first portion and the second portion differ in color.

7. The ultrasound imaging system of claim 6, wherein the indicator comprises a pair of boundary lines disposed on either side of the position of the viewable and non-viewable parts of the interventional device in the ultrasound image.

8. The ultrasound imaging system of claim 6, wherein the indicator comprises a path indicator disposed in the ultrasound image adjacent a tip of the interventional device and indicating a straight-line path within the object extending away from the tip.

9. The ultrasound imaging system of claim 1, wherein the detection and recognition system is configured to:
match one or more detected echogenic patterns to corresponding stored echogenic patterns in the memory unit;
determine the dimensions of the stored echogenic patterns from the memory unit;
determine the viewable and non-viewable parts of the one or more detected echogenic patterns by comparing the dimensions of the stored echogenic patterns with a representation of the one or more detected echogenic patterns in the ultrasound image data; and
position the indicator within the ultrasound image on the display.

10. An ultrasound imaging system comprising:
a processing unit configured to receive and process acquired ultrasound image data to create ultrasound images derived from ultrasound image data, the procession unit including a detection and recognition system configured to detect a pattern of echogenic features within the created ultrasound images;
a memory unit operably connected to the processing unit and storing information regarding individual interventional devices, including stored information regarding echogenic patterns and specific locations and dimensions of stored echogenic patterns on the individual interventional devices;
a display operably connected to the processing unit to present the ultrasound images to a user;
an ultrasound imaging probe operably connected to the processing unit to acquire the ultrasound image data for use by the processing unit to form the ultrasound images; and
an interventional device adapted to be inserted into an object adjacent the imaging probe, the interventional device including one or more echogenic portions thereon, the one or more echogenic portions including one or more echogenic features disposed thereon forming one or more echogenic patterns,
wherein the detection and recognition system is configured to detect one or more echogenic patterns within the ultrasound image data, to compare a detected echogenic pattern to the stored echogenic patterns within the memory unit and to position an indicator within an ultrasound image on the display illustrating a location of viewable and non-viewable parts of the interventional device in view of the positions of viewable and non-viewable parts of the detected echogenic pattern.

11. The ultrasound imaging system of claim 10, wherein the detection and recognition system is configured to:
match one or more detected echogenic patterns to corresponding stored echogenic patterns in the memory unit;
determine the dimensions of the stored echogenic patterns from the memory unit;
determine the viewable and non-viewable parts of the one or more detected echogenic patterns by comparing the dimensions of the stored echogenic patterns with a representation of the one or more detected echogenic patterns in the ultrasound image data; and
position the indicator within the ultrasound image on the display.

12. The ultrasound imaging system of claim 10, wherein the indicator comprises:
a first portion illustrating the position of viewable parts of the interventional device; and
a second portion illustrating the position of non-viewable parts of the interventional device.

13. The ultrasound imaging system of claim 12, wherein the indicator comprises a pair of boundary lines disposed on either side of the position of the viewable and non-viewable parts of the detected echogenic pattern in the ultrasound image.

14. The ultrasound imaging system of claim 12, wherein the indicator comprises a path indicator disposed in the ultrasound image adjacent a tip of the interventional device and indicating a straight-line path within the object extending away from the tip.

15. A method for providing an indication of viewable and non-viewable parts of an interventional device in an ultrasound image, the method comprising the steps of:
providing an ultrasound imaging system comprising:
a processing unit configured to receive and process acquired ultrasound image data to create ultrasound images derived from ultrasound image data, the procession unit including a detection and recognition system configured to detect a pattern of echogenic features within the created ultrasound images;
a memory unit operably connected to the processing unit and storing information regarding individual interventional devices, including stored information regarding echogenic patterns and specific locations and dimensions of stored echogenic patterns on the individual interventional devices;
a display operably connected to the processing unit to present the ultrasound images to a user;
an ultrasound imaging probe operably connected to the processing unit to acquire the ultrasound image data for use by the processing unit to form the ultrasound images; and
an interventional device adapted to be inserted into an object adjacent the imaging probe, the interventional device including one or more echogenic portions thereon, the one or more echogenic portions including one or more echogenic features disposed thereon forming one or more echogenic patterns,
wherein the detection and recognition system is configured to detect one or more echogenic patterns within the ultrasound image data, to compare a detected echogenic pattern to the stored echogenic patterns within the memory unit and to position an indicator within an ultrasound image on the display illustrating a position of viewable and non-viewable parts of the interventional device;
inserting the interventional device into the object;
obtaining ultrasound image data using the probe;
matching one or more detected echogenic patterns to corresponding stored echogenic patterns in the memory unit;
determining the dimensions of the stored echogenic patterns from the memory unit;
determining the viewable and non-viewable parts of the one or more detected echogenic patterns by comparing the dimensions of the stored echogenic patterns with a representation of the one or more detected echogenic patterns in the ultrasound image data; and
positioning the indicator within the ultrasound image on the display in alignment with the viewable and non-viewable parts of the one or more detected echogenic patterns on the interventional device.

16. The method of claim 15, further comprising the steps of:
re-determining the viewable and non-viewable parts of the one or more detected echogenic patterns by comparing the dimensions of the stored echogenic patterns with a representation of the one or more detected echogenic patterns in the ultrasound image data after positioning the indicator within the ultrasound image; and
altering the form of the indicator within the ultrasound image on the display in alignment with the viewable and non-viewable parts of the one or more detected echogenic patterns on the interventional device.

17. The method of claim 16, wherein the indicator includes a first portion aligned with the viewable parts of the one or more detected echogenic patterns and a second portion aligned with the non-viewable parts of the one or more detected echogenic patterns, and wherein the step of altering the form of the indicator comprises changing a length of at least one of the first portion or the second portion.

18. The method of claim 16, wherein the indicator includes a first portion aligned with the viewable parts of the one or more detected echogenic patterns and a second portion aligned with the non-viewable parts of the one or more detected echogenic patterns, and wherein the step of altering the form of the indicator comprises changing a color of at least one of the first portion or the second portion.

19. The method of claim 16, wherein the step of altering the form of the indicator comprises enhancing the indicator to identify a location of a tip of the interventional device.

20. The method of claim 18, wherein the step of matching one or more detected echogenic patterns to corresponding stored echogenic patterns in the memory unit comprises matching one or more detected echogenic patterns to a user-selected stored echogenic pattern.
